(19) United States
(12) Patent Application Publication
(10) Pub. No.: US 2019/0000572 A1
(43) Pub. Date: Jan. 3, 2019

(54) ROBOTIC ASSISTED PROSTATE SURGERY DEVICE

(71) Applicant: UNIVERSITY OF GEORGIA RESEARCH FOUNDATION, INC., Athens, GA (US)

(72) Inventors: Tsz Ho Tse, Lawrenceville, GA (US); Alex Squires, Athens, GA (US); Sheng Xu, Bethesda, MD (US); Reza Seifabadi, Bethesda, MD (US); Bradford Wood, Bethesda, MD (US)

(21) Appl. No.: 16/066,994

(22) PCT Filed: Dec. 29, 2016
(86) PCT No.: PCT/US16/69187

§ 371 Date: Jun. 28, 2018

Related U.S. Application Data
(60) Provisional application No. 62/273,674, filed on Dec. 31, 2015.

(51) Int. Cl. A61B 34/30; A61B 18/24; A61B 10/02; A61B 34/20

(52) U.S. Cl. CPC A61B 34/30; A61B 18/24; A61B 2018/00577; A61B 34/20; A61B 2010/0208; A61B 10/0241

(57) ABSTRACT

An example robot system for guiding a percutaneous device into a prostate of a patient includes a guide defining an opening configured to direct the needle, a robot coupled to the guide, and a rotation member coupled to the robot. The robot being configured to move the guide in a left-right and anterior-posterior directions based on information on the prostate of the patient. The rotation member being configured to change a yaw angle of the guide within a coronal plane of the patient.

### CROSS-REFERENCE TO RELATED APPLICATIONS

This application claims benefit of U.S. Provisional Application No. 62/273,674, filed Dec. 31, 2016, which is hereby incorporated by reference in its entirety.

### TECHNICAL FIELD

This disclosure relates to robotics based medical devices, and more particularly to robotic systems for guiding a needle into a prostate of a patient.

### BACKGROUND

Prostate cancer is the second leading cause of death from cancer in men. More than 200,000 men were diagnosed with prostate cancer in the U.S. in 2014 alone and nearly 30,000 died as a result of the disease that same year. The majority of diagnosed cases represent low-risk, organ-confined disease. The current definitive treatment for localized prostate cancer is radical prostatectomy which entails surgical removal of the part of, if not the entire, prostate. However, it is evident that prostatectomy over-treats the cancer leaving morbidities such as incontinence and sexual dysfunction. Alternative solutions to radical prostatectomy include active surveillance or localized treatment. Patients usually prefer the latter in order to avoid the anxiety of leaving the disease untreated.

Minimally invasive procedures have been explored to treat the prostate and avoid such morbidities. Focal laser therapy, for example, has emerged as a treatment alternative that can spare patients from many of these undesired side effects. Prostate focal laser ablation has been performed as a minimally invasive procedure to ablate tumors, using real-time MR thermometry to enhance safety near critical structures. Focal laser ablation (FLA) has the advantage of effectively treating the tumor volume while minimizing over-treatment in the surrounding tissues. Also, since it is done under MRI guidance, it benefits from accurate identification of the zone to be ablated.

MRI offers a superior imaging modality for prostate FLA for numerous reasons; first, it provides excellent visualization of the cancerous and healthy surrounding tissues. Secondly, it offers thermometry which entails real-time monitoring of the ablated zone. Last but not least, it provides real-time anatomical imaging that when combined with thermometry, provides enough information for safe ablation.

Other localized treatments for prostate cancer include cryo-ablation, and high-intensity focused ultrasound (HIFU).

Despite recent advances, improvements in the accuracy and efficacy of FLA (and other minimally invasive) procedures are still desired.

### SUMMARY

Provided are systems for guiding percutaneous devices into a prostate of a patient. An example system includes a guide defining an opening configured to direct a needle, a robot coupled to the guide, and a rotation member coupled to the robot. The robot being configured to move the guide in a left-right and anterior-posterior directions based on information on the prostate of the patient. The rotation member being configured to change a yaw angle of the guide within a coronal plane of the patient.

Another example system can be used for guiding a percutaneous device into a prostate of a patient in a MRI machine. The example system includes a guide defining an opening configured to direct a first end of a needle, a robot coupled to the guide, and a remote guide defining an opening. The robot being configured to move the guide in a left-right and anterior-posterior directions based on information on the prostate of the patient. The remote guide defining an opening configured to support a second end of the needle. The remote guide being configured to align with the guide and spaced a distance from the guide to for access outside a bore of the MRI machine. The system may also be used for CT, PET, or ultrasound guided procedures.

The details of one or more embodiments of the disclosure are set forth in the accompanying drawings and the description below. Other features, objects, and advantages of the disclosure will be apparent from the description, drawings, and from the claims.

### BRIEF DESCRIPTION OF THE DRAWINGS

FIG. 1 is a schematic of a robotic system for assisting minimally invasive surgery;

FIG. 2 is a schematic of a robot of the robotic system of FIG. 1 being used in an MRI machine;

FIG. 3 is an elevation view of a robot for directing a needle or catheter to a prostate;

FIG. 4 is a bottom plan view of the robot of FIG. 3 mounted on a base plate and attached to a rotation arm;

FIG. 5 is a schematic of a needle workspace using the robot of FIG. 4;

FIG. 6 is a perspective view of another robotic assistance system including a remote guide;

FIG. 7 is a side view of the robotic assistance system of FIG. 6;

FIG. 8 is a top view of the robotic assistance system of FIG. 6;

FIG. 9 is a front view of the robotic assistance system of FIG. 6;

FIGS. 10 and 11 are top views of the robotic assistance system of FIG. 6 with the rotation arm in different positions;

FIG. 12 is an exploded view of the remote guide and consumables from the robotic assistance system of FIG. 6;

FIG. 13 is top view of a catheter extension arm and instrument funnel of the robotic assistance system of FIG. 6;

FIGS. 14 and 15 are perspective views of the remote guide of the robotic assistance system of FIG. 6;

FIG. 16 is a perspective view of the robotic assistance system of FIG. 6 being employed in the bore of an MRI;

FIG. 17 is a set of linear graphs of an example count buffering algorithm implemented by the robotic assistance system of FIG. 1;

FIG. 18 is a set of plots showing the translation positioning accuracy of the robotic assistance system of FIG. 1 before and after implementation of the buffering algorithm of FIG. 17;

FIG. 19 is a side view of a needle guide channel without MRI-contrast agent embedded therein;

FIG. 20 is a perspective view of the needle guide channel of FIG. 19 without needle;

FIG. 21 is close up top view of the needle guide channel of FIG. 20 with MRI-contrast agent embedded therein;

FIG. 22 is an MRI image showing a front view of the needle guide channel of FIG. 21;

FIG. 23 is a front view of a freehand ball joint positioner of the robotic assistance system of FIG. 1, in which the view includes a needle guide in a first angular position;

FIG. 24 is another front view of a freehand ball joint positioner of the robotic assistance system of FIG. 1, in which the view includes a needle guide in a second angular position; and

FIG. 25 is another front view of a freehand ball joint positioner of the robotic assistance system of FIG. 1, in which the view includes a needle guide in a third angular position.

### DETAILED DESCRIPTION

Implementations of the present disclosure now will be described more fully hereinafter. Indeed, these implementations can be embodied in many different forms and should not be construed as limited to the implementations set forth herein; rather, these implementations are provided so that this disclosure will satisfy applicable legal requirements. As used in the specification, and in the appended claims, the singular forms “a”, “an”, “the”, include plural referents unless the context clearly dictates otherwise. The term “comprising” and variations thereof as used herein is used synonymously with the term “including” and variations thereof and are open, non-limiting terms.

The inventors have made the following observations regarding the prior art. During conventional FLA, a grid template (similar to a brachytherapy template) is used to guide the laser fiber (which delivers energy) into the targeted location under MRI-guidance. This template is suboptimal for several reasons. The distance among the holes is 5 mm, limiting the maximum accuracy. It does not provide needle angulation, which is sometimes required to avoid pubic arch interference or nerve bundles. It does not allow remote insertion of the needle; as a result, the patient has to be removed many times from the scanner bore, thus significantly increasing procedure time. The user interface included with the commercial product does not provide the capability and workflow required for effective treatment planning and subsequent procedures in FLA.

Although the devices and systems described below are illustrated facilitating use of FLA in a prostate treatment procedure in a magnetic resonance imaging (MRI) machine, the devices and systems can be employed in other procedures, on other anatomical structures and using a range of probes. For example, the devices and systems may be used for biopsy, cryoablation, and high-intensity focused ultrasound (HIFU). That being said, the disclosed devices and systems are particularly advantageous in the MRI setting and for use in prostate treatments or biopsies.

Generally, the robotic assistant system is a motorized, relatively compact, template-like robot with two degrees of freedom (DOF) that can guide a needle both in Anterior-Posterior and Left-Right directions with submillimeter accuracy. The robot also provides angulation (such as from −15 to +15 degrees) in the coronal plane to avoid nerve bundle interference. The workspace is designed to be large and variable and can have an hour-glass or dovetail shape. The system is easily attached to a patient board fixed to an MRI table and can quickly be registered to the MRI coordinate system with embedded fiducial markers. It is MRI compatible by using pressurized air for actuation and fiber optics for sensing. It is designed to mimic the design of surgical templates, thus minimizing divergence from current practices and can used manual insertion for maximum safety. The system can also include user-friendly software (e.g., OncoNav) designed for treatment planning and implementation of FLA and the software integrates with the clinical workflow required from image fusion, tumor segmentation, iterative ablation planning, MR thermometry monitoring, to post-treatment analysis.

As shown schematically in FIG. 1, a robotic assistant system 10 of one embodiment includes a control box 12, communication lines 14 and a robot 16. The control box 12 is positioned in a control room 18 and is connected to an air source 20 and a computer 22. The robot 16 is positioned in an MRI room 19, and in particular in a tubular opening of an MRI scanner 24, and is connected to the control box 12 via the communication lines 14. The computer 22, generally, is configured to collect data on scanned prostate images from the MRI scanner 24 and use that data to navigate the robot 16 to guide the location of a minimally invasive procedure, such as a biopsy or laser ablation of the prostate.

The control box 12 is configured for controlling the motion of the robot 16 and can include, for example, one or more air valves 68, a data acquisition board 70 and a receiver 72. The air valves 68 are connected via a non-metallic line 74 to the robot 16 (for MRI compatibility) and connect also to the air source 20. The air valves 68 are configured to modulate the amount of air supplied to the motors of the robot 16 to adjust the position of the robot as described in more detail below. Also, the receiver 72 is connected via an optical fiber line (dashed) 76 to optical encoders on motors of the robot 16 to detect the position of its components. These positions are fed back to the receiver 72, processed by the data acquisition board 70 and then communicated over a conventional metallic line 78 to the computer 22 for further processing in the context of the MRI scanner data. A health care person can interact with the computer 22 to accurately implement desired treatment or biopsy protocols.

The communication lines 14 are configured to have non-metallic components in the MRI room 19 to avoid interference with the operation of the MRI scanner 24, such as by use of the air line 74 and the optical fiber line 76. The components of the communication lines 14 in the control room 18, on the other hand, are shielded and may include conventional lines such as a the metallic line 78.

It should be noted that although particular configurations of control hardware and software are described herein, the data collection and control systems can be implemented using a range of local and distributed hardware, software and firmware and still accomplish the desired objectives of automatic or semi-automatic control or assistance for a minimally invasive procedure. Also, other types of scanners may be employed to determine the anatomy used to guide the operation of the robotic system 10—such as ultrasound or CT scans.

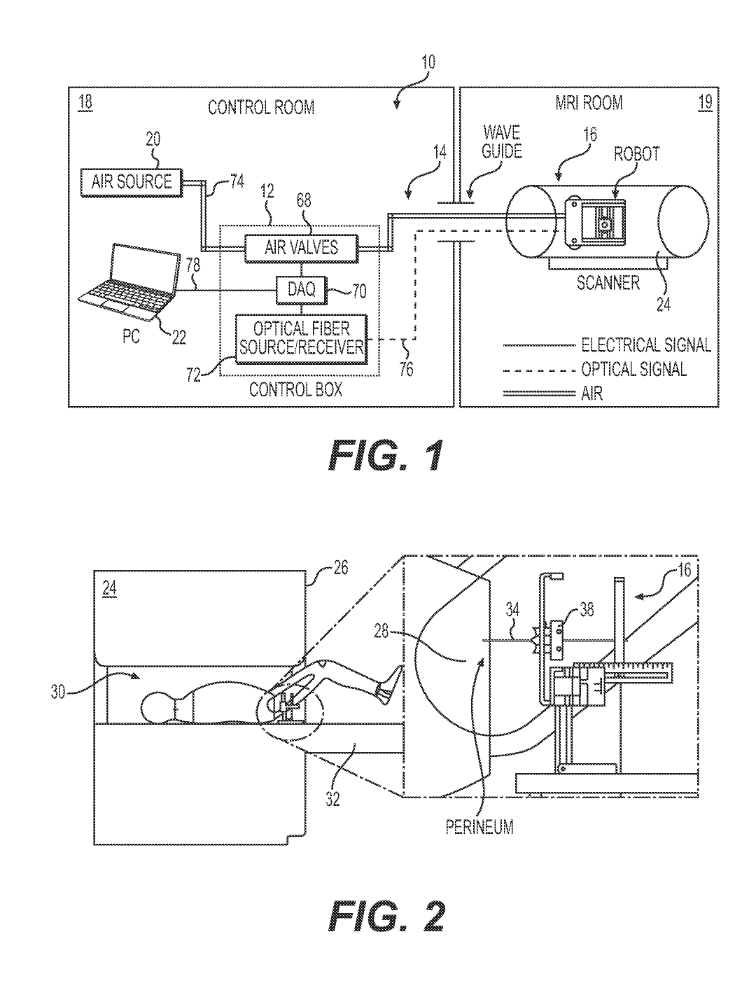

As shown in FIG. 2, a portion of the robot 16 is positioned in the opening of the MRI scanner 24 and adjacent a perineum 28 of a patient 30. The patient 30 is positioned on a table 32 with upper thighs and the remaining superior portion (head, shoulders, torso, etc.) of the patient in the opening of the MRI scanner 24—inside a plane defined by the edge of the opening in the gantry, a.k.a., a gantry plane 26. Extending out past the gantry plane 26 are the bent knees of the patient 30 and the lower legs and feet of the patient held up by a support (not shown). The table 32 can be translated in and out of the MRI opening, past the gantry plane 26, to position the patient within the MRI scanner 24 for scanning.

The robot 16 is positioned on the table 32 inside the MRI opening, past the gantry plane 26, in between the legs of the patient. The robot 16 uses a guide 38 to guide a biopsy or ablation catheter delivery needle 34 through a perineum 28 of the patient. As will be described more below, the robot 16 can position the guide 38 in an anterior-posterior (with respect to the patient) and left-right planes based on anatomical information from the MRI. The needle 34 itself is manually advanced by the health care worker through the perineum and into the prostate, thus providing tactile feedback and avoiding the hazards of a closed-loop robotic system.

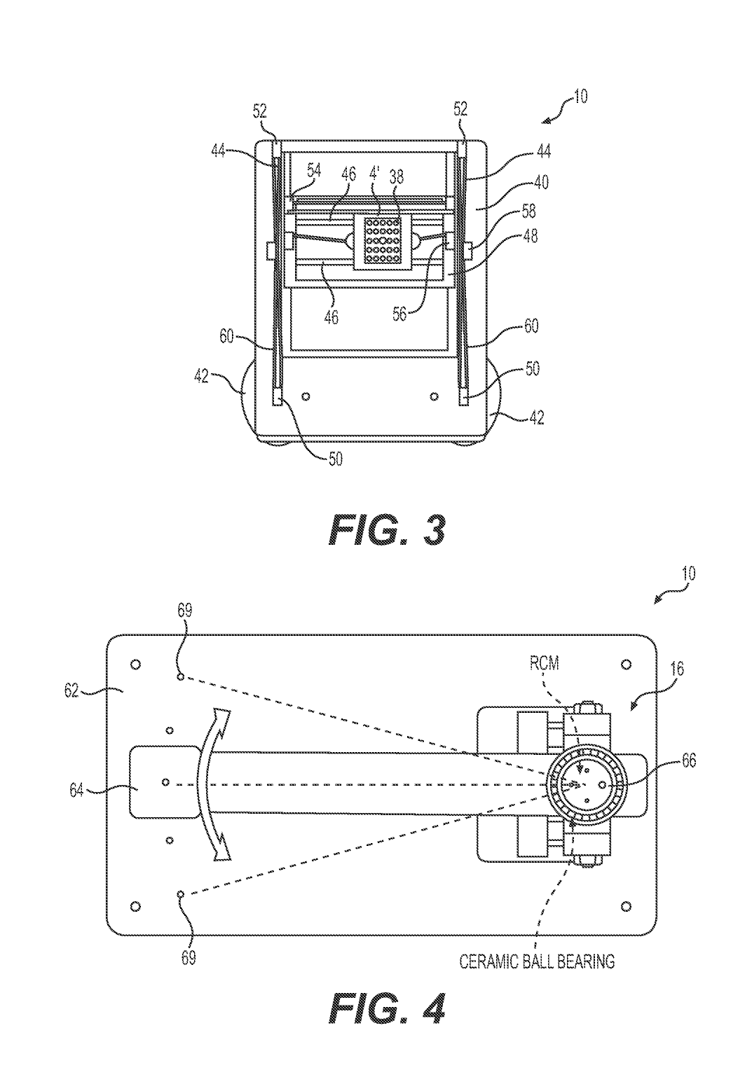

The robot 16 of one embodiment configured to continuously move, with two motors, the needle guide 38 in two degrees of translation. In the transverse plane, the robot can offer (for example) 51 mm of horizontal (left-right) movement and 83 mm of vertical (ventral-dorsal or anterior-posterior) movement. As shown in FIG. 3, the robot includes a frame 40, a pair of motors 42 and the needle guide 38.

The frame 40 is rectangular and includes a height (in the anterior-posterior direction) of about 135 mm and a width (in the left-right direction) of about 140 mm. The frame 40 defines a rectangular opening within which are supported a pair of vertical bars 44 and a pair of horizontal bars 46. The vertical bars 44 extend from the top to the bottom of the opening on its lateral sides and are fixed with respect to the frame 40. The horizontal bars 46 extend perpendicular to the vertical bars 44 and are slidably supported thereon by a sub-frame 48. The needle guide 38 is laterally, slidably supported via its own frame on the horizontal bars. The needle guide 38 has a rectangular shape with multiple holes in a grid-like pattern through which a needle can be slid to pierce the perineum.

The motors 42 are supported partially on a back surface of the frame 40 at its lower left and right hand corners. The motors 42 are preferably MRI-compatible, such as pneumatic motors with optical encoders. The motors 42 have shafts mated to and driving a pair of motor-driven pulleys 50. The frame 40 also supports top pulleys 52 at the top corners of the rectangular frame opening and sub-frame 48 supports a first pair of pulleys 54 and a second pair of pulleys 56. The frame 40 may also support belt tension adjusters 58.

A continuous belt 60 runs from the outside motor-driven pulleys 50 up to the top pulleys 52, down to the first pulleys 54, turning at a right angle to connect across the rectangular opening above the needle guide 38. The belt 60 runs from the inside of the motor-driven pulleys 50 up to the second pulleys 56 on the sub-frame 48 and have ends connecting to respective left and right sides of the needle guide 38. The belt tension adjusters 48 are configured to slide laterally in slots in the frame 40 to adjust the tension in the continuous belt 60.

By nature of its pathway over the pulleys, the continuous belt 60 can be moved by equal rotations (clockwise or counterclockwise) of the motor-driven pulleys 50 to adjust the anterior-posterior position of the sub-frame 48. Differentials in the amount of movement of the motor-driven pulleys 50 also serves to make left-right adjustments of the needle guide 38. The motors 42 also include optical encoders which provide feedback to the control box 12 on the movement of the motor-driven pulleys 50 to determine the relative location of the needle guide 38 to the initial, referenced MRI image of the patient's prostate. Advantageously, the anterior-posterior range of the robot 16 is about 83 mm and the left-right range is about 51 mm, with a resolution of less than 0.1 mm.

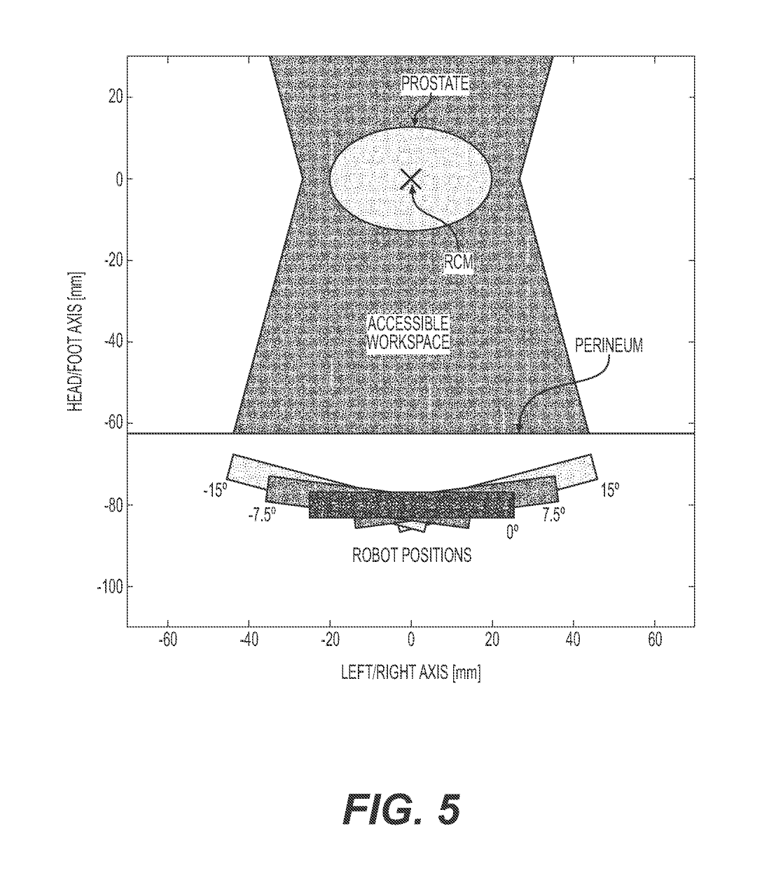

As shown in FIG. 4, the robot 16 can be mounted on a base plate assembly which can include a base plate 62, rotation arm 64 and a bearing 66. The base plate 62 has a rectangular shape with a long-axis extending in the general direction of needle advancement. The base plate can be affixed to the MRI table—such as via grooves on the side of the MRI table. The width of the robot permits the robot to face against the patient's perineum while the end effector is less than 25 mm away from the perineum.

Defined in the base plate 62 are a plurality of index holes 69. The base plate 62 also supports the bearing 66 on an end opposite (along the long axis of the base plate) the index holes 69. The index holes 69 are arrayed in 7.5 degree increments about an arc with a center positioned coincident with a center of rotation of the bearing 66. The bearing 66 supports a bottom of the robot 16's frame.

The rotation arm is positioned between the bearing 66 and the frame 40 and extends away from the frame. The rotation arm has an elongate rectangular shape and extends generally along the long axis of the base plate 62 back to the index holes 69. The end of the rotation arm 64 defines an opening through which a pin or other indexing device can be passed to register the arm with one of the index holes 69. Thus, the rotation arm and bearing establish a discrete pivotal DOF that pivots the robot (and the needle guide 38) about a remote center of motion (RCM), providing 0°, ±7.5° and ±15° relative to 0° or parallel to the axis of the MRI bore.

Advantageously, the RCM can be positioned under the prostate. And, use of the RCM allows the robot and needle guide to be located at a different location from the pivot point while still rotating about the prostate and providing optimal coverage of the desired volume, i.e. the prostate. It should be noted that the rotation about the RCM could be smaller or larger angles, or even be continuous, such as by being positioned on a continuous arc-shaped track. Convenience, usability and rigidity of fixation are achieved, however, through the use of indexing the rotation arm. The RCM can also be positioned inferiorly or superiorly of the prostate, such as by 20 mm, 40 mm or 60 mm.

As shown in FIG. 5, the rotation arm 64 and its ability to rotate about bearing 66 around the RCM, and the prostate, creates an hourglass-shaped workspace in two dimensions for the end of the needle or probe. (Not all of the workspace is shown in FIG. 5 as it extends in the superior and inferior direction to the limits of the reach of the needle or probe.) Also, the anterior-posterior adjustability of the needle guide 38 allows this same hourglass shape to be accessed in multiple levels in the anterior-posterior direction. Thus, the 2-D hourglass shapes stack up in the anterior-poster direction to form a 3-D dovetail shape where the angled sides form into angled, intersecting planes when viewed from the perspective of FIG. 5. The shape of this workspace facilitates targeting of tumors located laterally to the urethra and neurovascular bundles without damaging these critical structures. Thus, the robot system 10 can provide coverage of the prostate comparable to, or exceeding that of, existing systems, with continuous coverage in the transverse plane and along the scanner axis via adjustable insertion depth.

It should be noted that the term “hourglass shape” is used to define any two-dimensional shape with left and right edges that generally converge as they extend toward a narrower waist (which can be positioned at the prostate) and then generally diverge out again extending away from the waist. The hourglass need not be symmetrical on the left or right side, nor do the side edges need to be linear. Also, the superiorly-directed divergence need not have the same angle, width or extent of the inferiorly-directed divergence. With that being said, the illustrated hourglass shape of FIG. 5 has symmetry superior and inferior of the prostate, left and right symmetry, and equal 15 degree angles on the left and right sides.

The effective workspace of the robot 16 is adjustable by varying the distance between the robot and the RCM as well as the relationship between the prostate and the RCM. The robot can be situated directly on top of the rotating arm or away (at 0, 20, 40, or 60 mm for example) from the RCM, permitting adjustable distance between the robot and the perineum. The RCM can be situated directly under the prostate or slid towards the head or feet by moving the robot board. The combination of these two settings allows for optimal coverage of the prostate as each patient can be given a custom configuration. In the scenario of the patient's prostate being situated directly over the RCM, the robot offers full coverage at all angulations. In a scenario where the RCM lies inferior to the prostate, some loss of coverage is found at the edges of the prostate in the widest angulation positions. But, the inner positions still offer full coverage. Angulation is primarily used to avoid anatomical structures along the center line of the body, further diminishing the loss of any lateral workspace coverage.

It should also be noted that there are other automated linkages (fully or partially automated) that can provide the hourglass shaped workspace. Any x-y translation mechanism, for example, could be placed on an arc-shaped track with a center positioned at the RCM to line up with the prostate. Also, the limitations to the hourglass shape need not be physical, they can be due to software limits, for example. There are robots with full 6 degrees of freedom that can mimic the same workspace shape, such as through choice of a coordinate system and appropriate software-based stops on the angles through which the end-effector holding a probe would pass. The challenge, though, with most robots will be having an MRI compatible system within reasonable cost-constraints.

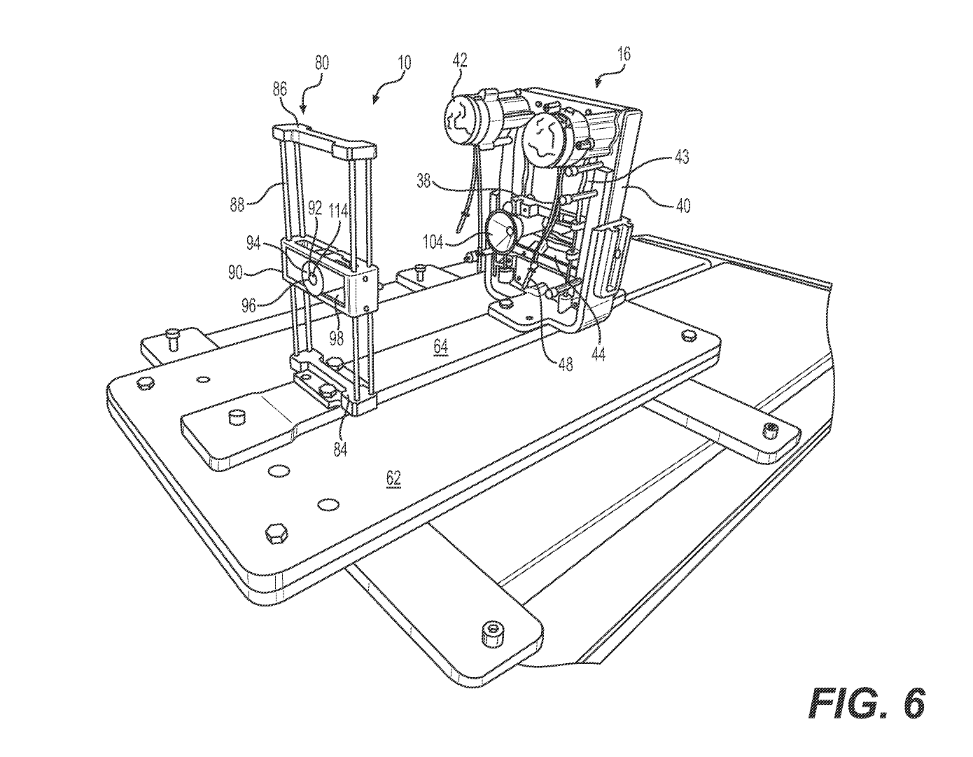

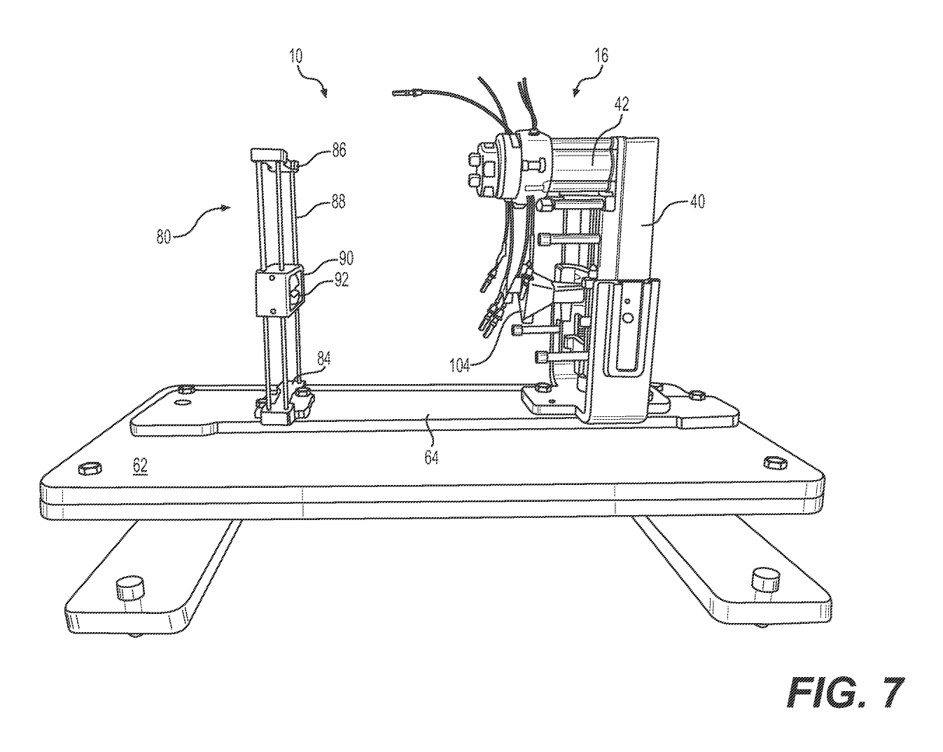

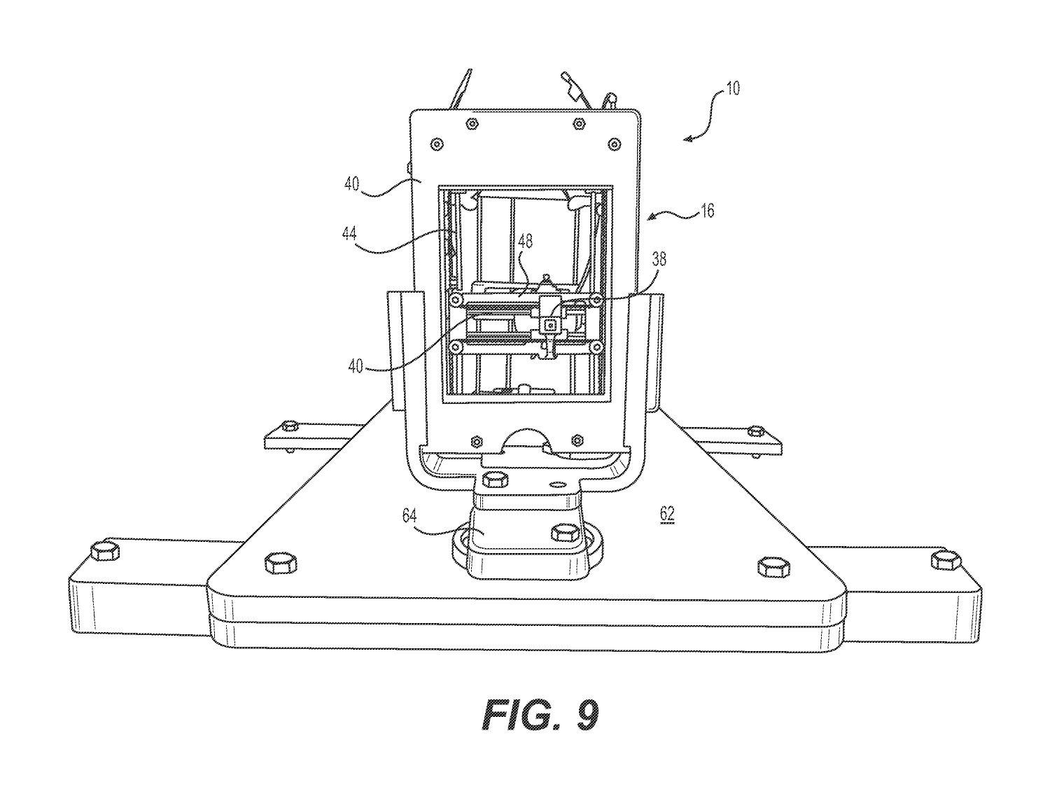

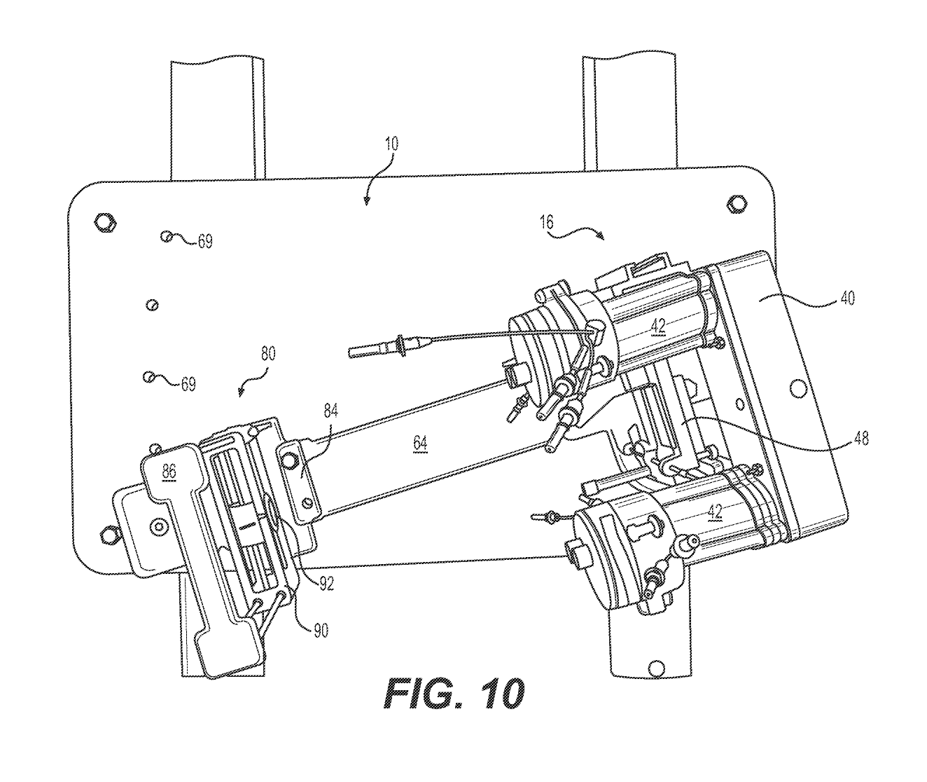

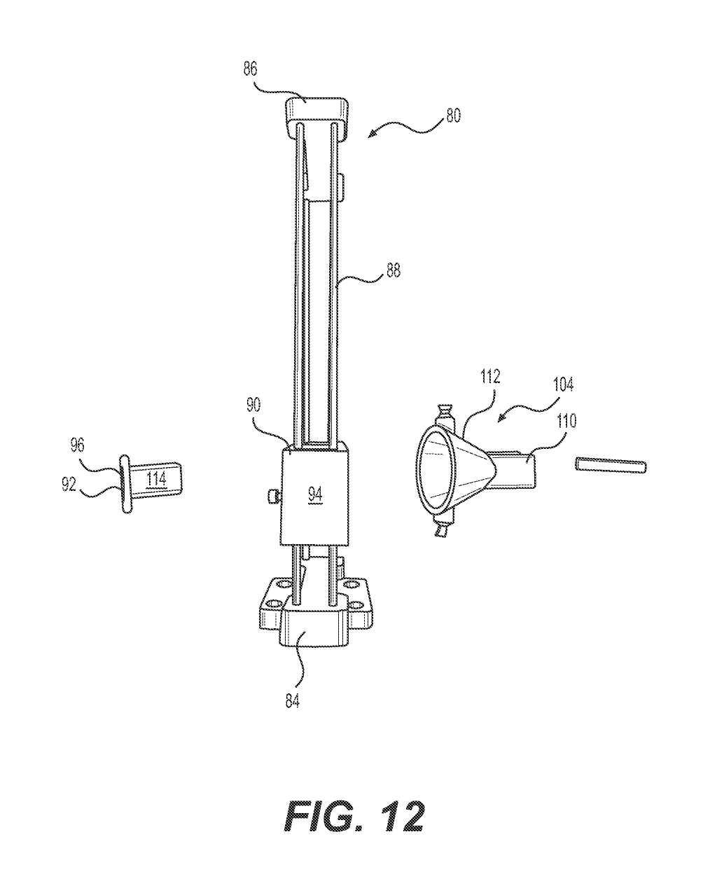

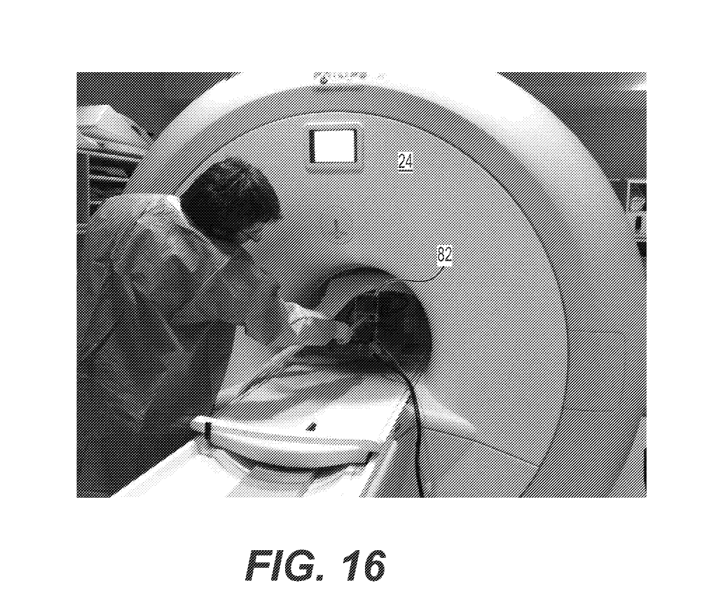

FIGS. 6-16 show another embodiment of the robotic assistant system 10 that includes a remote guide 80 for positioning outside of the gantry plane 26. Generally, the remote guide 80 provides a support for use of an elongate catheter extension arm 82. The catheter extension arm has sufficient length to allow the healthcare worker to stand outside of the bore of the MRI scanner 24 and still advance the needle 34 through the guide 38 of the robot 16 and into the prostate of the patient 30, as shown in FIG. 16. This is a much more comfortable position for the healthcare worker and allows repositioning of the needle 34 without removing the patient from the bore of the MRI scanner 24. The remote guide 80 minimizes the number of patient removals from the scanner, enabling real-time visual feedback (as the healthcare worker can still see a screen) during insertion and reducing the procedure time and cost.

Referring again to FIG. 6, the remote guide 80 is attached at its base near the indexed end of the rotation arm 64 opposite the robot 16. The remote guide 80, generally, is manually matched to the robot 16's position and facilitates insertion of the needle or catheter from outside the MRI scanner 24's bore. The remote guide 80 includes a base plate 84, top plate 86, vertical rods 88, slider 90 and instrument channel 92.

The base plate 84 has a rectangular shape and is configured to attach to the rotation arm 64 and support, via cylindrical openings, a set of four of the vertical rods 88 in an upright, vertical orientation (extending anterior-posterior with respect to the patient). At the top end, the top plate 86 has a similar rectangular structure within which are secured the tops of the vertical rods 88. The slider 90 has a rectangular frame defining through holes near its left and right edges to allow it to be mounted, and slide upon, the four vertical rods 88. Defined in the slider 90 is a rectangular slot with its long axis oriented in the left-right direction.

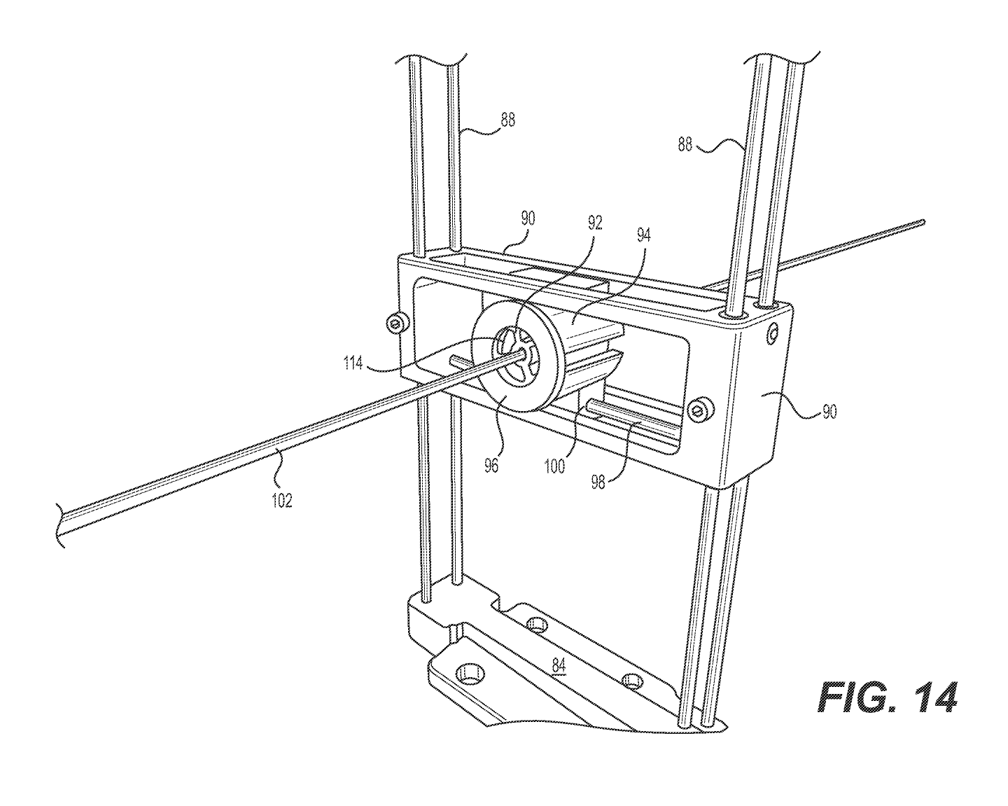

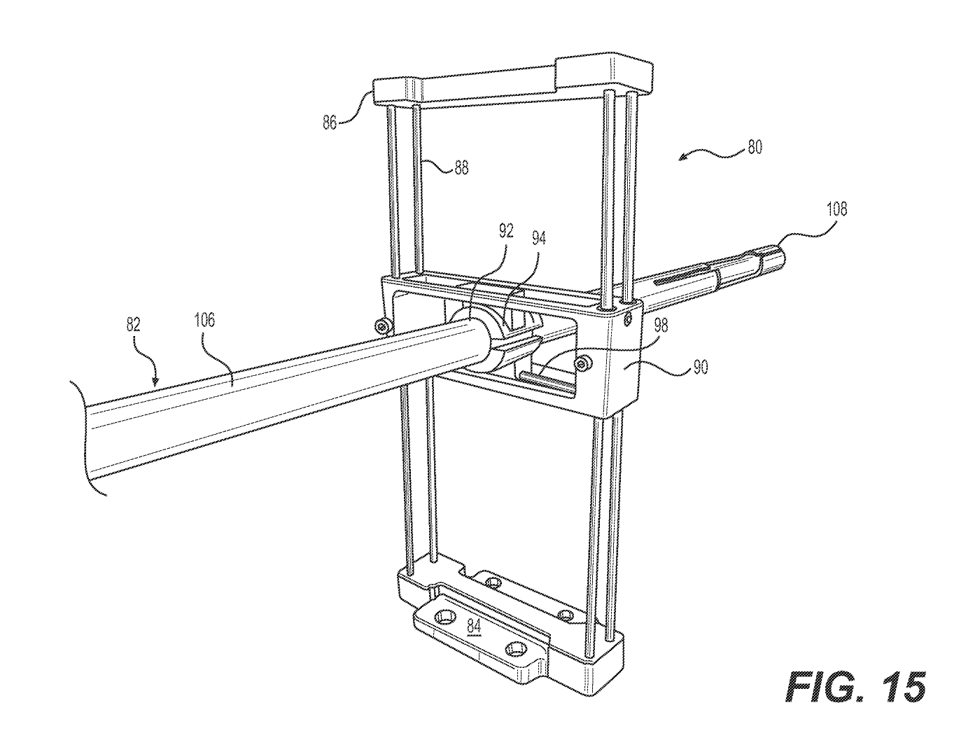

The instrument channel 92, as shown in FIGS. 14 and 15, has a rectangular outer housing 94 defining a cylindrical opening 96 extending therethrough. The outer housing 94 is configured to slide within the rectangular opening of the slider 90 along a pair of horizontal rods 98. The outer housing 94 in particular includes mounting bores 100 extending laterally along the top and bottom of the outer housing. The horizontal rods 98 pass through these bores 100 and support the lateral movement the instrument channel 92 within the slider 90.

The cylindrical opening 96 can be filled with a valve structure 114 that accommodates a smaller diameter catheter 102 for guiding the needle 34 and/or ablation probe, as shown in FIG. 14. Also, the valve structure 114 may expand accommodate the larger diameter catheter extension arm 82, as shown in FIG. 15, for guiding needles and dilators to form the hole in the patient's perineum 28. The catheter extension arm 82 may be removed after placement of the needle, dilator or catheter to leave the smaller diameter tube residing in the valve structure 114, as shown in FIG. 14.

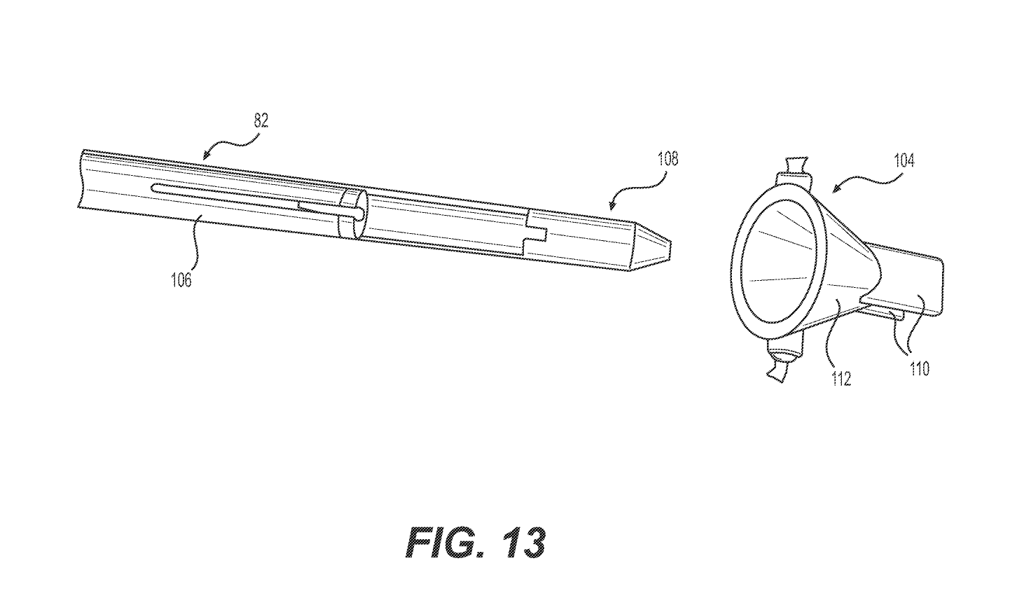

FIG. 13 shows components of a consumable assembly, including the catheter extension arm 82 and an instrument funnel 104 meant to facilitate the origination of the hole in the patient's perineum using the remote guide 80 and the robot 16. (The term “consumable” meaning components that can be easily and cheaply removed and disposed of without sterilization after contact with biological materials.)

The catheter extension arm 82 includes an elongate tubular structure 106 and a conical tip 108. The elongate tubular structure 106 has a wide bore defined by a relatively stiff tubular wall structure that can be easily gripped and advanced through the instrument channel 92 to the robot 16. The conical tip 108 has an outer conical surface that corresponds with an internal conical surface of the instrument funnel 104.

The instrument funnel 104 includes a pair of parallel mounting walls 110 and a funnel portion 112. The mounting walls 110 are parallel wall structures extending from the narrow end of the funnel portion 112. The funnel portion 112 has a conical shaped wall structure defining a converging, conical shaped opening that is congruent to the conical tip 108 of the catheter extension arm 82.

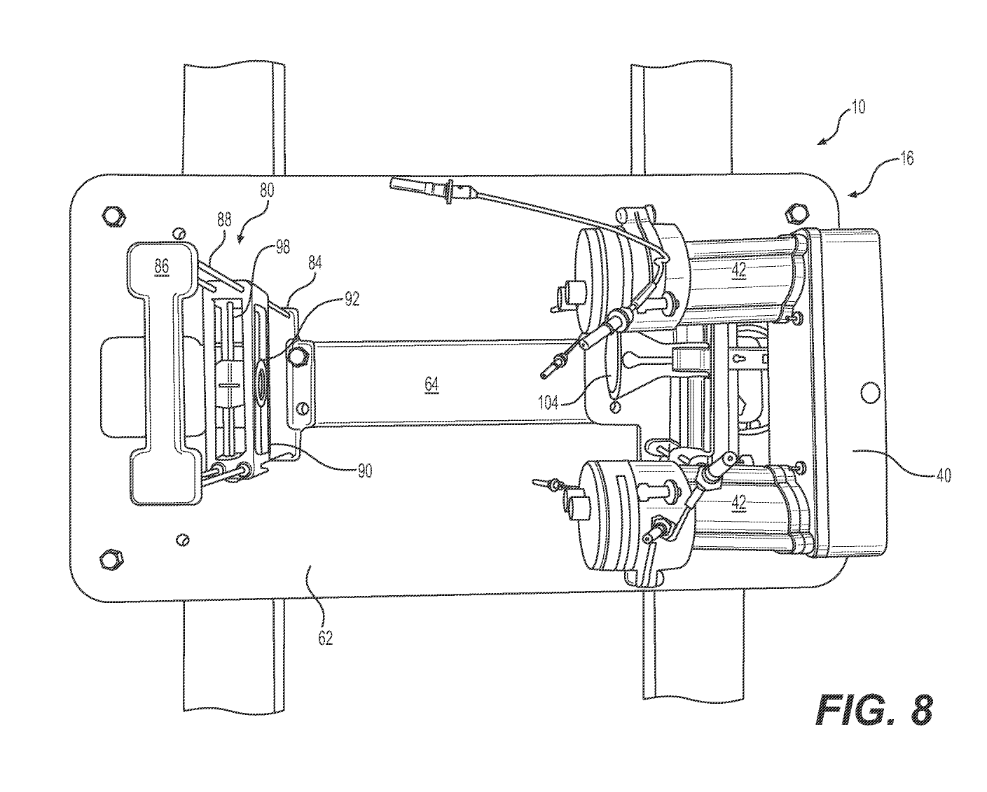

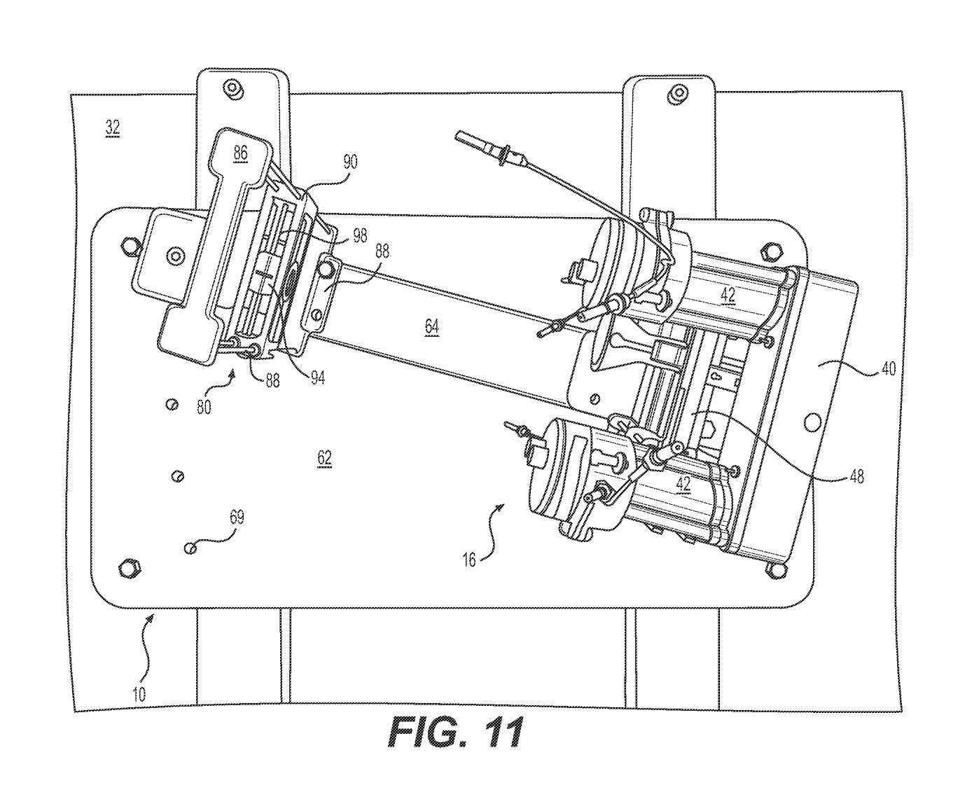

As shown in FIGS. 8 and 11, the instrument funnel 104 can be mounted to the subframe 48 of the robot 16 by extending the parallel wall structures around a portion of the subframe. The conical shaped opening may converge to a small diameter through-hole for precise direction of the needle 34 therethrough. The instrument funnel 104 could also be mounted to the guide 38 in registration with one of its holes to guide the needle 34. Regardless, the funnel portion 112 helps to urge the advancing conical tip 108 into alignment with the through-hole defined at the base of the funnel portion 112, even when being advanced from a remote location at the remote guide 80 by the healthcare worker.

During use, after the computer 22 and control box 12 have directed the robot 16 to align the guide 38 and/or instrument funnel 104 with the desired biopsy or treatment site, the health care worker takes the catheter extension arm 82, adjusts the anterior-posterior and left-right positioning of the instrument channel 92 to approximate the location of the instrument funnel 104, and advances the catheter extension arm through the valve structure 114 until it reaches the funnel portion 112, as shown in FIG. 16. Continued advancement into the funnel portion 112 urges the distal conical tip 108 into position and thereby adjusts the left-right and anterior-posterior positioning of the instrument channel 92.

The health care worker then advances a needle or dilator through the bore of the catheter extension arm 82 and through the instrument funnel 104 to the desired depth within the patient's prostate. The health care worker retracts and removes the catheter extension arm 82. The health care worker then advances the delivery catheter 102 over the needle or dilator, and removes the needle or dilator leaving the catheter in place. Subsequently, biopsies or treatments can be applied through the delivery catheter 102.

The robotic assistant system 10 can include several advantages. The motors are both fixed, simplifying the design of the robot and avoiding obstructing the workspace. The motors enhance the physical rigidity and enable optical quadrature encoding. Quadrature encoding integrated into the design of the motor allows for precise positioning and control of the robot in the transverse plane. The guidance channel 104 and its valve structure 114 can be removed from the robot for sterilization as well as use of alternate sized channels for differing needle gauges.

Advantageously, the hardware and software system disclosed herein for prostate laser ablation treatment uses image data of the patient's prostate soft tissue in high resolution. MRI is currently the most useful imaging modality for producing the required high-resolution images of the prostate. Therefore, the proposed system benefits from access to MRI scanners in order to perform laser ablation delivery.

The robot 16 can be controlled through a graphical user interface, such as an interface designed in LabVIEW 2014 (National Instruments, Austin, Tex.). The graphical display demonstrates the workspace, end effector position, and target position. Operators can choose to drive the end effector to the target position either manually or automatically. Automatic control can be performed using a positioning-seeking algorithm on each of the motors. When a target point is entered, the necessary rotation for each motor is calculated and used as the goal position. The control scheme is based on proportional control with a deadband of 0.3 mm. The ramping of speed associated with proportional control can cause quick movements to close the distance to the target and more precise tuning of position as the target is approached.

Selection of a target point can be performed in different ways. For example, an operator can enter an absolute point or the program can calculate a target point based on changes in position relative to the robot's current position. Based on the target point, the program gives a tool length to which the needle, catheter, or other tools should be inserted.

The graphical user interface can draw its data, for example, from OncoNay. OncoNav is a Java-based software platform developed at the NIH for image guided interventions. In robotically assisted FLA, OncoNav directly communicates with the MRI scanner, allowing MRI images to be displayed and processed during or immediately after each scan. At the beginning of the intervention, a T2 weighted scan of the robot is acquired. The fiducials embedded in the robot are manually identified in the image, which are used to register the robot to the MRI scanner. After that, a high resolution scan of the prostate is taken to identify and segment the tumor. During the ablation, the software monitors the temperature of FLA and calculates the real-time temperature map using the proton resonance frequency shift (PRFS) method. Since the temperature map has very little anatomic information, the software overlays the temperature map on the corresponding planning T2 weighted image, which provides real-time verification of the treatment plan.

The ablation zone of the laser fiber is fairly small. Multiple ablations are often needed to treat a large lesion. In one scenario, the lesion should be fully covered with the least number of ablations while the collateral damage to the healthy tissue is minimized and all the critical structures near the lesion are protected. This is a numerical optimization problem with conditions. Accurate modeling of the shape and size of each ablation zone facilitates prediction of the outcome of the composite ablations. The complexity of the mathematical model has an impact on the computation time of the optimization. Unlike other ablation options such as radiofrequency ablation (RFA), the laser ablation zone has the shape of a prolate ellipsoid with a very sharp boundary, allowing it to be modeled using equation (1)

$${( \frac{x}{p} )}^{2} + {( \frac{y}{p} )}^{2} + {( \frac{z}{q} )}^{2} = 1 \qquad ( 1 )$$

where the semi-axes are of lengths p, p, and q with q>p. The size of the ablation zone is a function of ablation time. Since the prostate does not have major blood vessels, the heat sink effect is small, making the ablation zone highly predictable.

In order to reduce the computation time and avoid suboptimal solutions, a multiresolution scheme can be implemented. The ablation zone model is first rotated so that the longest principal axis is aligned with the later fiber. The algorithm starts at the coarsest resolution. At each resolution level, both the tumor and the ablation model are digitized to the corresponding image resolution. The locations of the ablations are optimized using the Powell method to maximize the combined coverage of the tumor. If multiple solutions have the same coverage of the tumor, the solution with the lowest collateral damage to healthy tissue will be selected as the best solution. This result is used to initialize the optimization at the next finer resolution level. For a selected number of ablations, the output of the algorithm is an array of ablation locations in the diagnostic image and their coverage of the tumor. The final plan is the plan with the least number of ablations and 100% coverage of the tumor.

The treatment plan in the diagnostic image can be used to guide multiple ablations to achieve optimal tumor coverage without the need for scanning the patient in between each individual ablation, therefore significantly reducing the procedure time.

The robotic assistance system 10 utilizes multi-parametric MRI to optimally target and monitor tumors during composite laser ablations. The robotic positioner hardware improves the ease and speed of the needle placements and reduces unnecessary gland punctures. This system addresses the unmet clinical need for real-time, additive, composite information on where the multiple laser effects have been, and where tumors still need treatment. The tumor image can fused to the ablation zone and the two compared during the procedure, until there is no more untreated tumor tissue. The robotic positioner simplifies clinical workflow by optimizing access to the prostate tumors, enhancing laser catheter positioning accuracy and consistency, and reducing the procedure time.

The robot-assisted MRI-guided FLA demonstrates high accuracy in needle positioning, provides needle angulation and remote insertion capabilities.

Experimental testing was performed to evaluate the robotic assistance system 10 disclosed herein when used with FLA. The ablation of desired target volume was successful with minimal damage to healthy surrounding areas. A targeting error mean of 0.46 mm (SD=0.25 mm) was recorded in open air tests. Seed placements errors had a mean of 0.9 mm (SD=0.4 mm) perpendicular to the needle guide, and a mean of 1.9 mm (SD=2.7 mm) along the needle guide. The ablation procedure covered 100% of the virtual tumors and presented mild spillover from the ablation zone.

In some embodiments, the control box 12 of the robotic assistance system 10 is further configured to implement a buffer algorithm to reduce the amount of error caused by gear backlash and mistightened timing belts upon motor reversal. Gear backlash and fractionally mistightened timing belts can cause a motor upon reversing to effectively jump ahead of the end-effector as motion is lost as a result of the gear train and belts reversing into the backlash behind the previous direction of actuation.

The control box 12 can compensate for the issue above by establishing a buffer on each axis comprising a buffer value (Bₙ) that is a quadrature count that accounts for the backlash translation caused by reversing motor direction. In particular, the receiver 72 of the control box 12 can be configured to receive and forward an input count (dCᵢₙ) to a buffer algorithm from the quadrature encoder of the motor. The buffer algorithm generates an output modified count (dCₒᵤₜ) that can be used by the data acquisition board 70 to update the position of the robot 16. After each loop, the buffer algorithm updates the buffer value (Bₙ) from the previous loop to create a new buffer value (Bₙ₊₁) that based on the motor quadrature count of the current loop (dCᵢₙ). This period process of updating is embodied in the formula below:

Bₙ₊₁=Bₙ+dCᵢₙ

In the case where the present buffer value (Bₙ₊₁) of a given loop is greater than a maximum buffer value (Bₘₐₓ), the buffering algorithm generates an output quadrature count (dCₒᵤₜ) comprising the difference between the maximum buffer value (Bₘₐₓ) and the present buffer value (Bₙ₊₁). The maximum buffer value (Bₘₐₓ) can be determined in a variety to suitable ways, including for example, by utilizing Aurora EM tracker or microscribe digitizer to empirically track the lost distance or angle caused by motor reversal, and then converting the result to a quadrature count. In the case where the present buffer value (Bₙ₊₁) is less than 0, the buffer algorithm generates a quadrature count (dCₒᵤₜ) that is equal to the present buffer value (Bₙ₊₁). In the case where the present buffer value (Bₙ₊₁) falls between 0 and the maximum buffer value (Bₘₐₓ), the buffer algorithm generates an output quadrature count (dCₒᵤₜ) that is equal to 0. The following is a mathematical expression that describes the generation of an output quadrature count (dCₒᵤₜ) by the buffer algorithm in the manner described above:

if Bₙ₊₁>Bₘₐₓ:dCₒᵤₜ=Bₙ₊₁−Bₘₐₓ

elseif Bₙ₊₁<0:dCₒᵤₜ=Bₙ₊₁

else: dCₒᵤₜ=0

After performing the above, the buffer algorithm then limits the present buffer value (Bₙ₊₁) to be within the range of [0, Bₘₐₓ] before passing the altered buffer value (Bₙ₊₁) to the next loop. For example, in the case where both the maximum buffer value (Bₘₐₓ) and the present buffer value (Bₙ₊₁) are positive, the buffering algorithm sets the new buffer value (Bₙ₊₁) to be the value of the present buffer value (Bₙ₊₁) or the maximum buffer value (Bₘₐₓ), whichever is lower. In contrast, in the case where the maximum buffer value (Bₘₐₓ) or the present buffer value (Bₙ₊₁) is negative, the buffering algorithm sets the new buffer value (Bₙ₊₁) to 0. The following is a mathematical expression that describes the altering of the buffer value (Bₙ₊₁) by the buffer algorithm in the manner described above:

Bₙ₊₁=max(0,min(Bₙ₊₁,Bₘₐₓ))

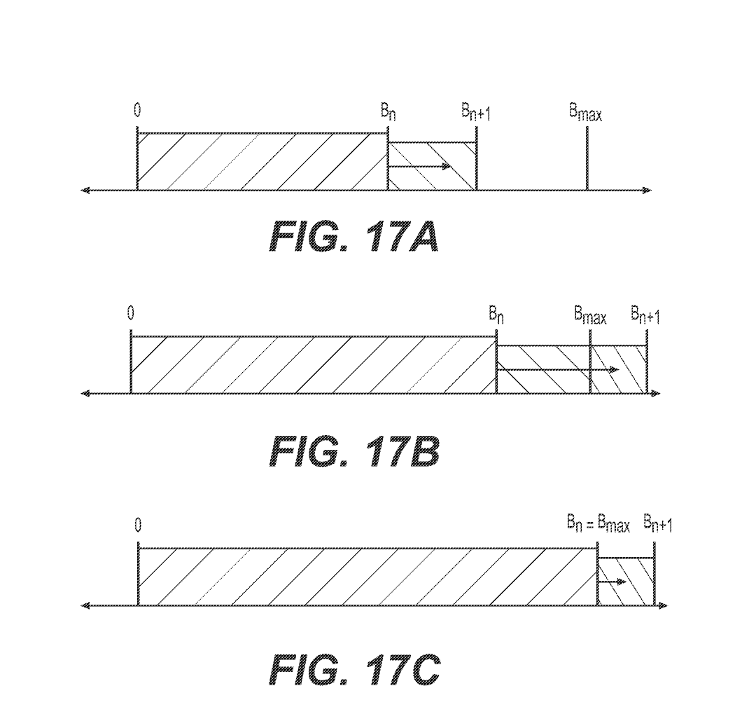

FIG. 17 shows an example of three buffering scenarios in accordance with the buffering algorithm described above. In a first scenario (a), the present buffer value (Bₙ₊₁) falls between 0 and the maximum buffer value (Bₘₐₓ), thus no count is passed by the buffering algorithm to the data acquisition board 70 for distance calculations. In a second scenario (b), the present buffer value (Bₙ₊₁) exceeds the maximum buffer value (Bₘₐₓ), thus the difference between the two values is passed by the buffering algorithm to the data acquisition board 12 for distance calculations. In a third scenario (c), once the buffer limit has been reach, the count in its entirety is then passed to the data acquisition board 70 for distance calculations.

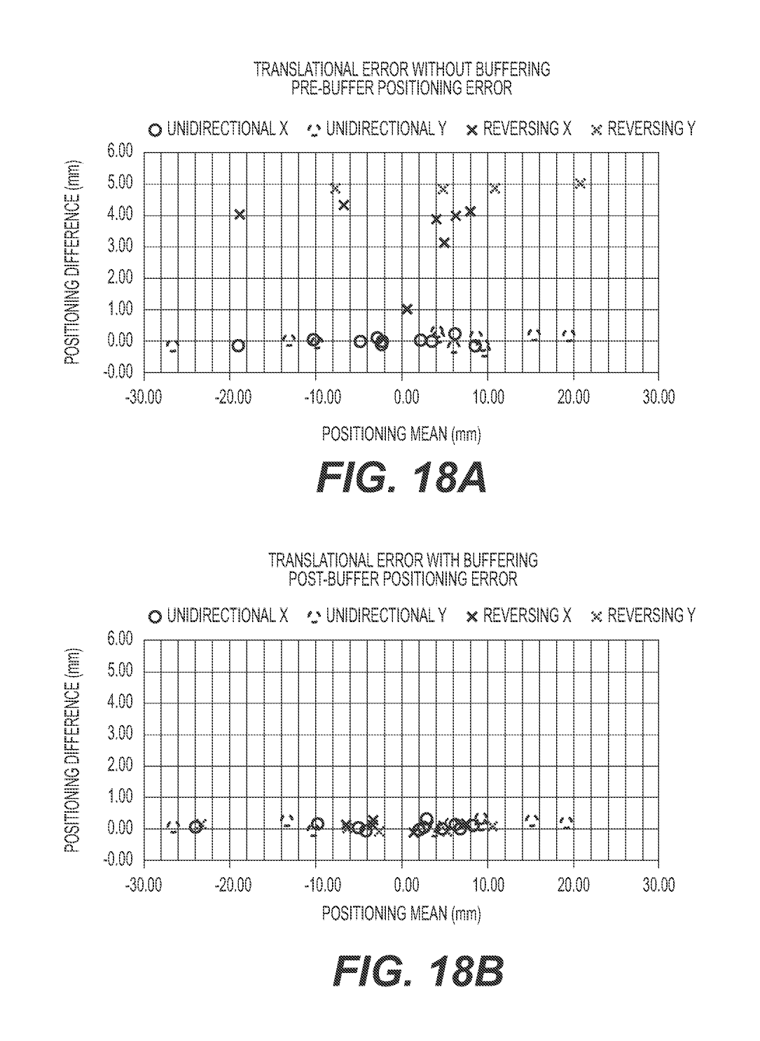

FIG. 18 shows a set of plots that represent the translation positioning accuracy of the robotic assistance system 10, before and after implementation of the buffering algorithm, as measured by one empirical study that was conducted by the investors. During the test the reported and actual positions were recorded with the mean plotted on the horizontal axis and the difference on the vertical axis. As shown in the first plot (a) of FIG. 18, consistent errors were clearly visible when an axis reversed direction by the motor prior to implementation of the buffer algorithm. Two smaller errors were visible for reversal on the x-axis, but analysis of the data revealed that these points were subsequent test points during which the end effector did not move during the first data point. Summation of the two points placed the result in the same range as other x-axis reversal errors. As shown in second plot (b) of FIG. 18, the buffer implementation disclosed herein was found to substantially remove the systemic error. A two-sample t-test confirming the efficiency of the buffering algorithm described herein as the test rejected the null hypothesis that reversal and unidirectional actuation were statistically different (p=0.61).

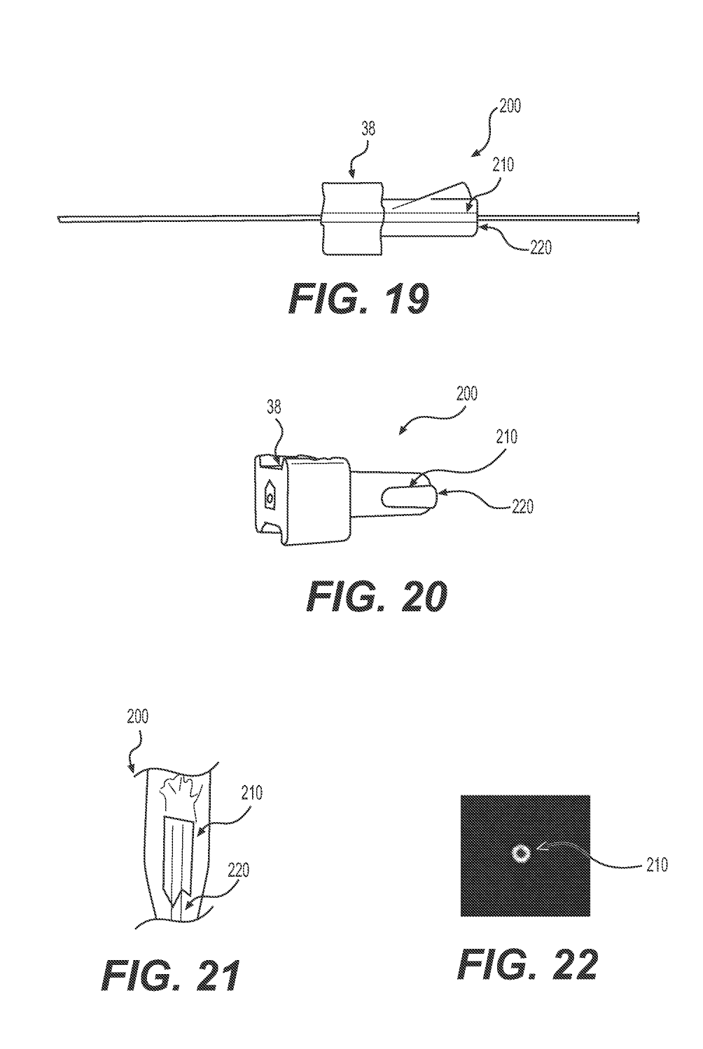

In some embodiments, fiducial markers need not be embedded on the robot at fixed location in order to register the robot to the MRI scanner. Rather, in some embodiments, the robotic assistance system 10 includes a needle guide channel 200 having fiducial fluid embedded therein to register the robot 16 to the MRI scanner. As shown in FIGS. 19-22, inn one embodiment the needle guide channel 200 comprises a needle guide 38 having a hollow cavity 210 that is wrapped around a portion of a needle channel 220. The needle channel 220 defines a through-hole that is sized to receive the needle 34. The hollow cavity 210 is filled with a fiducial fluid, such as an MRI-contrast, for example, which can be used to calculate the location of the needle guide 38 in the MRI coordinate system. FIG. 22 shows an MRI image of what is seen when the example needle guide channel 200 is scanned by an MRI scanner. The fiducial fluid can be obtained from various suitable sources, including for example, through extraction of fiducial fluid from a commercial fiducial marker. The needle guided channel 200 can be formed using a variety of suitable manufacturing processes including, for example, 3D printing. Formlabs Form 2 3D-printer is one example of a suitable 3D printer.

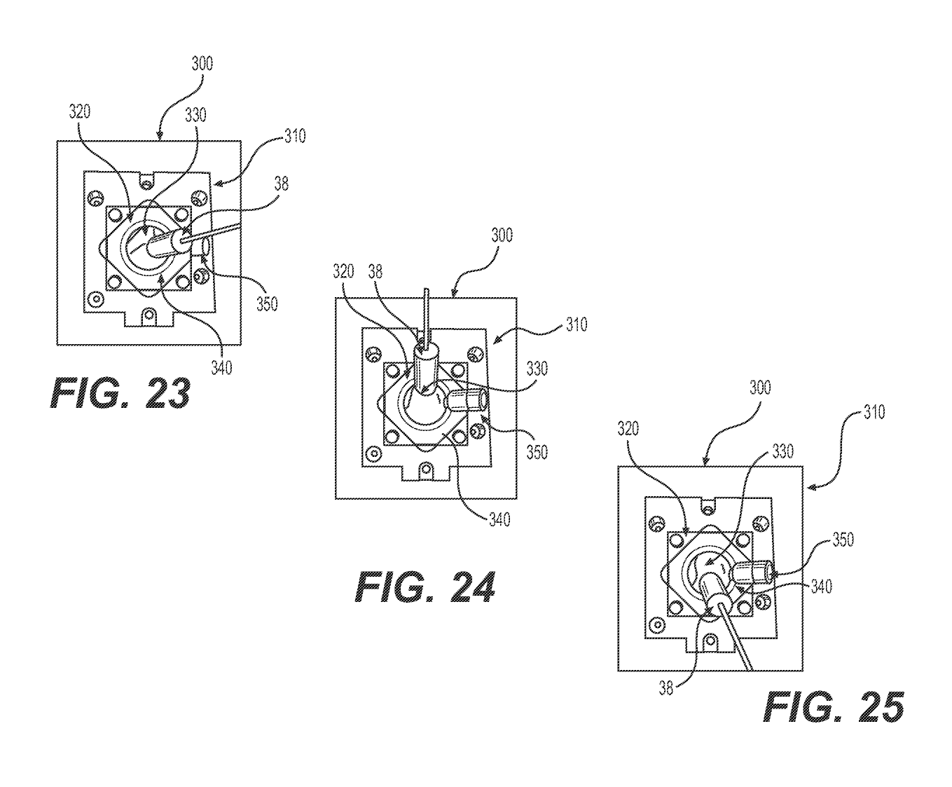

In some embodiments, a ball joint positioner 300 may be added to the end-effector of the robotic assistance system 10 to allow for fine angulation positioning of the end-effector. FIGS. 23-25 show one such example of a ball joint positioner 300 comprising a top and bottom concentrically aligned structures 310, 320, in which the top and bottom structures 310, 320 include a spherical cavity 320 located therebetween which retains the needle guide 38 in a ball-in-socket configuration. The spherical cavity 330 is sized such that the need guide 38 can float and be turned to various suitable angles as allowed by the geometry of the top rectangular structure 310. The bottom structure 320 includes a hole 340 that is sized to accommodate a range of suitable angles for the positional angulation of the end-effector. After the needle guide 38 is positioned at a desired angle, a set screw 350 is used to hold the needle guide 38 in place via friction. In some embodiments the top and bottom structures 310, 320 are rectangular, circular, or square shaped.

The above-described MRI-compatible, robotic assistance devices and systems have several advantages. For example, it guides the needle to the desired location based on a priori MR images with superior accuracy, angulation and remote insertion capabilities. The improved accuracy can reduce the number of MRI scans during multiple composite ablations, thus shortening the procedure time. The robotic assistant removes the burden of needle guidance of the physician thus making the procedure more efficient and straightforward. All a physician needs to do is to insert the needle to the prescribed depth. The devices and systems avoid negatively influencing MR image signal to noise ratio (SNR), procedure workflow, bulkiness, and patient safety by making the needle orientation fully automatic.

A number of aspects of the systems, devices and methods have been described. Nevertheless, it will be understood that various modifications may be made without departing from the spirit and scope of the disclosure. Accordingly, other aspects are within the scope of the following claims.

That which is claimed:

1. A robotic system for guiding a needle into a prostate of a patient, the system comprising:
a guide defining an opening configured to direct the needle;
a robot coupled to the guide and configured to move the guide in a left-right and anterior-posterior directions based on information on the prostate of the patient; and
a rotation member coupled to the robot and configured to change a yaw angle of the guide within a coronal plane of the patient.

2. The system of claim 1, wherein the rotation member is configured to change the yaw angle about a remote center of motion.

3. The system of claim 2, wherein the remote center of motion is positioned with an axis extending through the prostate.

4. The system of claim 2, wherein the remote center of motion is positioned with an axis inferior or superior to the prostate.

5. The system of claim 2, wherein the yaw angle is +/−15 degrees with respect to a sagittal plane of the patient.

6. The system of claim 5, wherein the rotation member includes a bearing positioned at the remote center of motion.

7. The system of claim 6, further comprising a base plate, wherein the rotation member is mounted via the bearing to the base plate and wherein the base plate is configured to be mounted to a table of an MRI.

8. The system of claim 7, wherein the robot is supported above the bearing on one end of the rotation member and the rotation member has another end configured to extend out of a bore of the MRI.

9. The system of claim 2, wherein the robotic system defines a working envelope for the needle having an hourglass shape in the coronal plane.

10. The system of claim 9, wherein the hourglass shape is symmetrical in the coronal plane.

11. A robotic system for guiding a needle into a prostate of a patient in a MRI machine, the system comprising:
a guide defining an opening configured to direct a first end of the needle;
a robot coupled to the guide and configured to move the guide in a left-right and anterior-posterior directions based on information on the prostate of the patient; and
a remote guide defining an opening configured to support a second end of the needle, the remote guide configured to align with the guide and spaced a distance from the guide to for access outside a bore of the MRI machine.

12. The system of claim 11, wherein the remote guide includes a frame supporting an instrument channel for movement in the left-right and anterior-posterior directions, wherein the instrument channel defines the opening.

13. The system of claim 12, wherein the frame includes vertical rods on which a slider frame can slide in the anterior-posterior direction.

14. The system of claim 13, wherein the slider defines a slot within which the instrument channel can slide in the left-right direction.

15. The system of claim 11, further comprising a catheter extension arm configured to pass through the opening of the remote guide and the opening of the guide, the catheter extension arm including a tubular body and a conical tip defining a tip hole.

16. The system of claim 15, wherein the guide includes an instrument funnel, the instrument funnel configured to receive the conical tip and guide the conical tip into a known location with respect to a plurality of fiducial markers of the robotic system.

17. The system of claim 16, wherein the instrument funnel includes a mounting portion configured to engage a frame of the robot.

18. The system of claim 17, wherein the frame of the robot supports at least one motor.

19. The system of claim 18, wherein the guide, robot and remote guide are constructed of MRI compatible materials.

20. The system of claim 11, further comprising a rotation member having an elongate shape with a first rotatable end supporting the robot and guide and a second, opposite end supporting the remote guide and wherein the rotation member is configured to rotate in a coronal plane of the patient.
